(19) United States
(12) Patent Application Publication
(10) Pub. No.: US 2020/0129048 A1
(43) Pub. Date: Apr. 30, 2020

(54) ELASTOMERIC SHEATH FOR ENDOSCOPE AND METHOD OF MANUAFACTURING THE SAME

(71) Applicant: Canon U.S.A., Inc., Melville, NY (US)

(72) Inventors: Mark Alan Hamm, Lynnfield, MA (US); Jacob Schieffelin Brauer, Cambridge, MA (US)

(21) Appl. No.: 16/174,753

(22) Filed: Oct. 30, 2018

(51) Int. Cl. A61B 1/00; A61B 1/07

(52) U.S. Cl. CPC A61B 1/00135; A61B 1/00096; A61B 1/0014; A61B 1/00066; A61B 1/07

(57) ABSTRACT

A medical apparatus such as a catheter or endoscope that has a stretchable sheath is provided. The medical apparatus comprises an elongate core, a sheath substantially surrounding the elongate core, and a window attached to the distal end of the sheath. The sheath comprises an elastomeric section and is concentrically disposed and is configured to allow the length of the sheath to conform to the length of the elongate core and may also comprise a semi-rigid section.

### FIELD OF THE DISCLOSURE

The subject disclosure relates to medical apparatus such as endoscopes used in a medical and/or biological setting. Moreover, the subject disclosure relates to apparatus, their manufacture and assembly including endoscopes with sheaths having an elastomeric section.

### BACKGROUND INFORMATION

In the field of endoscopes, particularly those that employ a rotating core and separate non-rotating portion of the endoscope, numerous technological challenges exist, as discussed herein below:

(i) A first challenge is how to minimize unwanted light transmitted from the illumination optical fiber to the detection optical fiber,
(2) A second challenge is how to keep the rotating optics (illumination) in ideal position with respect to the distal imaging window to facilitate optimal imaging, including maximized field of view, and
(3) And a third challenge is how to minimize the force exerted on the imaging window during operation, to protect both the window and also the illumination optics from damage and from piercing through the imaging window of the sheath.

And more specifically, a challenge is how to keep the distal optics in ideal position very near the window without them touching the window, which could break or damage the optics and possibly damage the imaging window. For imaging catheters that employ a rotating, flexible drive cable, a conformal sheath that stretches to conform to minor length variations without a large force should be used. This allows the imaging core to rotate while pressed against the imaging window while the sheath conforms to minor length variations generated from bending the device during handling.

One proposed solution to the aforementioned optical challenges is a “stretchable sheath” used in conjunction with medical endoscopes, catheters and probes or the like. A survey of the prior art indicates that stretchable sheaths have not yet been adapted or designed with an objective of keeping the distal optics in ideal position very near the window without them touching the window, which could break or damage the optics and possibly damage the imaging window. In support of the above statement/observation, several prior art examples pertaining to stretchable sheaths and medical devices will now herein be discussed below.

A first prior art example of a stretchable sheath utilized in the medical field is taught in U.S. Pat. No. 6,270,494, entitled “Stretchable Cryoprobe Sheath” to Kovalcheck et al. (hereinafter “KOVALCHECK”). In KOVALCHECK, a sheath is disclosed for use on a closed loop Joule-Thomson cryosurgical probe, and the combination of the sheath and the closed loop probe. The sheath is slipped over the probe, thereby separating the probe from the environment. The sheath has a grip which fits over the handle of the cryosurgical probe, and an extendible shroud which can be longitudinally extended to cover the probe and which are attached to the handle. The sheath has a hollow multi-lumen catheter shaped and sized to fit snugly over the cannula of the cryosurgical probe. The catheter is not thermally conductive, preventing transfer of heat from the ambient to the gas mixture, and preventing the freezing of tissues at undesired locations along the catheter. A thermally conductive cap or tip is attached to the distal end of the hollow catheter. The thermally conductive cap or tip is biased against the cold tip on the probe by a biasing element in the sheath assembly, to promote heat transfer. (See KOVALCHECK Abstract).

While KOVALCHECK discloses a stretchable sheath equipped with conductive and non-conductive segments to transmit or selectively block the transmission of heat to the anatomy. However, KOVALCHECK is particular to cryogenic probes, having thermally conductive and non-conductive areas, and covers the handle as well. Moreover, KOVALCHECK further does not address the need for an optical window having a controlled thickness.

A second prior art example of a stretchable sheath utilized in the medical field is taught in U.S. Pat. No. 6,007,482, entitled “Endoscope with Stretchable Flexible Sheath Covering” to Madni et al. (hereinafter “MADNI”). In MADNI, an endoscope is provided which is both flexible and easily cleaned having a pair of telescoping sections at its distal end one of which carries a camera and which are alternately actuated to provide movement through a body passageway by a Bowden type of cable. Such cable has an outer helical casing with an inner steel wire. Respectively attached to the two cylindrical sections are inflatable bladders which provide for the movement above which also are an integral part of the flexible sterilized sheath being held to the respective sections by O-rings. (See MADNI Abstract).

While MADNI provides a conformal sheath that has a pair of telescoping section that allow movement of the articulating, telescopic core, such technology utilizes corrugated sections that are intended to be inflated to actuate movements within the body. The underlying endoscope has multiple segments that expand and contract longitudinally to move the scope tip through the anatomy, so the sheath needs to be very flexible to allow the inchworm type of motion within the GI tract. Moreover, MADNI further does not address the need for an optical window having a controlled thickness.

A third prior art example of a stretchable sheath utilized in the medical field is taught in U.S. Pat. No. 6,623,449, entitled “Catheter with Up-Going and Down-Going Configurations” to Paskar (hereinafter “PASKAR”). In PASKAR, a transformable catheter includes a sheath having a length which is a substantial fraction of the entire length of the transformable catheter and a bore there through. A preformed inner catheter having a complex curve formed into the distal end thereof is disposed in the sheath bore. By axially moving the inner catheter with respect to the sheath, various tip shapes may be achieved. At least one wire runs from the proximal end of the sheath to the vicinity of the distal end of the sheath to selectively deflect the distal tip of the sheath as desired by the user to assist in reformation and transformation of the tip of the catheter. By suitable manipulation of the wire and of the inner catheter with respect to the sheath, the shape of the exposed portion of the distal end of the inner catheter may be transformed to any of a variety of shapes. (See PASKAR Abstract).

While PASKAR teaches a conformal sheath to permit the distal portion of the device to conform to the desired shape depending on which part of the anatomy one wishes to access, this conformal elastic sheath is designed to accommodate various tip shapes depending on where the user wants to inner component to go within the vasculature. Only the distal segment is elastic and flexible to allow steerage through various tortuous anatomies. Moreover, PASCAR further does not address the need for an optical window having a controlled distance from the optics of the device.

Thus, a known disadvantage common to KOVALCHECK, MADNI and PASKAR and other proposed solutions is that none of these prior art references teach about an optical window of controlled distance from the optics. That is to say, a problem still exits with the above proposed solutions regarding how to keep the distal optics in ideal position very near the window without them touching the window, which could break or damage the optics and possibly damage the imaging window. For imaging catheters that employ a rotating, flexible drive cable, a conformal sheath that stretches to conform to minor length variations without a large force is needed. This allows the imaging core to rotate while pressed against the imaging window while the sheath conforms to minor length variations generated from bending the device during handling.

Since there is still a known disadvantage common to the above prior art references and other proposed solutions regarding an optical window of controlled thickness, a need continues to exist for improvement such optical windows having controlled thicknesses. In view of these considerations, it would be advantageous to address and/or overcome at least some of the deficiencies described herein above.

Accordingly, it would be advantageous to provide a stretchable sheath for endoscopes and method of manufacturing the same that addresses and/or overcomes at least some of the deficiencies indicated herein above.

### SUMMARY OF THE DISCLOSURE

According to various example embodiments of the present disclosure, a sheath having an elastomeric section (e.g., a stretchy sheath) for endoscopes and method of manufacturing the same is disclosed that addresses and/or overcomes at least some of the deficiencies indicated herein above.

One aspect of the disclosure addresses the challenges with a stretchable sheath designed and/or optimized to maintain the distal optics in ideal position very near the window without the distal optics touching the window, which could break or damage the optics and possibly damage the imaging window. The stretchable sheath covers, for example, most of the length of the device with an elastomeric section that comprises a tube that is attached to the low-profile, distal portion of the device but is incrementally shorter than the rotating core, and it conforms to minor length variations during operation to effectively minimize the forces that the rotating core exerts on the distal imaging window of the sheath during operation.

According to an embodiment of the present disclosure, a medical apparatus is provided having an elongate core having a distal and proximal end. This elongate core may include, for example, an imaging optical core and may require rotation. The elongate core may also include fibers and a drive cable. A sheath substantially surrounds the elongate core, where the sheath comprises an elastomeric section concentrically disposed around the elongate core. The elastomeric section is configured to allow the length of the sheath to conform to the length of the elongate core. A window is attached to the distal end of the sheath, wherein the distal end of the elongate core is near the window. The window may be simply attached, e.g. via adhesive or it may be embedded in the distal end of the sheath.

The sheath may comprise a semi-rigid section as well as the elastomeric section. Preferably, the elastomeric section is proximal to the distal end of the sheath (e.g., just proximal to a multi-lumen connecting the elongate core to, for example, a light source and/or a detector.)

In some exemplary embodiments, the elastomeric section of the sheath comprises a stretchable thermoplastic elastomer and may be made, for example, by extrusion from one of PEBAX, silicon rubber or polyurethane. The sheath may be formed from, for example, polyimide tubes with PTFE low-friction surface on the inner diameter of the tube plus low-friction coatings on the components that are in contact with the window during operation. The elastomeric section may comprise only a portion of the sheath. In some embodiments, the sheath further comprises a semi-rigid section. In some embodiments, the elastomeric section of the sheath is near the distal end of the apparatus, such as just proximal to the multi-lumen and the semi-rigid section of the sheath is located at the usable/deployable distal portion of the apparatus.

In some exemplary embodiments, the elongate core comprises a rotatable imaging core, such as an OCT imaging core described, for example, in U.S. Pat. Nos. 7,872,759; 8,289,522; 8,676,013; 8,928,889; 9,557,154 and U.S. Pat. Pub. 2017/0135584; and WO 2016/015052 and may also include an additional modality.

The sheath may be configured to allow the length of the sheath to conform to the length of the elongate core. With the window attached to the distal end of the sheath, this allows for the window to be touching the distal end of the elongate core. Alternatively, the window may be maintained a set distance from the elongate core (e.g., approximately 0.05 mm, approximately 0.1 mm, approximately 0.2 mm, etc.). Thus, the window can be maintained at such a distance when images are being obtained, such as during the pull-back in an OCT image acquisition and/or acquisition of an additional modality such as fluorescence imaging. In other alternative embodiments, the interaction between the window and the distal end of the elongate core can be defined by the contact force. Preferably, the contact force between the window and the elongate core is low. Thus, the force is 1 gm to 1000 gm or more particularly 1 gm to 100 gm or even more particularly 1 gm-10 gm.

In some exemplary embodiments, an endoscope is provided. The endoscope comprises an elongate core having a distal and proximal end, wherein the elongate core comprises an illumination fiber and a distal optics at the proximal end of the elongate core; a sheath substantially surrounding the elongate core, the sheath comprises an elastomeric section which is concentrically disposed around at least a proximal portion of the elongate core and an inner positioned tube which is concentrically disposed around the elongate core; and a window attached to the distal end of the sheath, wherein the distal optics near the window.

According to an embodiment of the present disclosure, an endoscope is provided that includes a multilumen extrusion having a hollow center including, a first extrusion segment; a second extrusion segment having a smaller diameter than the first extrusion segment; and a third extrusion tapered segment interconnecting the first extrusion segment and the second extrusion segment. A plurality of satellite lumens are formed along the entire length of the multilumen extrusion such that each of the plurality of satellite lumens is circumferentially positioned an equal radial distance from a center axis of the multilumen and equally spaced apart from each other. An imaging window is integrally disposed within a distal end of the second extrusion. A stretchable sheath is provided which includes an outer tube connected to a proximal end of the first extrusion segment via adhesive, melt-bond or the like. Moreover, an inner tube is included with at least one coiled wire formed therein a tubular wall of the inner tube substantially extending the entire length of the endoscope and concentrically positioned within the hollow center of the multilumen extrusion and a hollow center of the outer tube, the inner tube having a distal end being secured within the second extrusion segment, and wherein a remaining portion of the inner tube not secured within the second extrusion segment being freely floating within the third extrusion tapered segment, first extrusion segment, and within the entire length of the outer tube. A plurality of detection fibers extend the entire length of the endoscope, each one of the plurality of detection fibers respectively disposed within a respective one of the plurality of satellite lumens. Additionally, an elongate core is concentrically disposed within the inner tube substantially extending the entire length of the endoscope. The imaging core includes a rotatable illumination fiber; at least one coiled wire circumferentially coiled around the inner tube such that the at least one rotatable coiled wire is fixedly secured to the inner tube. A bumper is concentrically disposed at the distal end of the rotating imaging core. The bumper includes a distal bumper portion having a distal surface pressed against an internal side of the imaging window and further including a distal optics receiver; and a proximal bumper portion contiguously connected to the distal bumper portion adapted to fixedly receive distal ends of the rotatable illumination fiber the at least one rotatable coiled wire. Furthermore, an optical element having a distal optical surface and proximal optical surface, the optical element configured to be secured within the distal optics receiver such that the distal optical surface is held at a predetermined distance from the internal side of the imaging window.

And, according to still another embodiment of the present disclosure, a ferrule affixed to a proximal end of the illumination fiber; and a spring loaded optical connector configured to receive the ferrule, wherein when the optical connector is attached to a mating connector, the ferrule compresses the spring, thereby effectively lengthening the rotating image core by approximately 0.10 mm to 2.0 mm, or more particularly, 0.10 mm to 0.70 mm. This is useful because the product may sit on the shelf for over a year prior to its use, so if the stretchable sheath were to remain under tension the entire time, it would likely permanently stretch, thereby reducing or eliminating the small force holding the distal optics near the imaging window. This would also overcome a change in the distance between the optics and the window that is due to differing coefficients in thermal expansion between the core and the sheath when the scope heats up inside the body. This is particularly relevant for long scopes (e.g. 2-3 meter scopes for gastroenterology).

Furthermore, according to yet another embodiment of the present disclosure, the multilumen extrusion is extruded with larger lumens and outer diameter and then drawn down to a final diameter using a heated drawing die, or shrunk using heat shrink tubing to a final outer diameter using heat and tension, to size the outer diameter, again with heat.

These and other aspects, features, and advantages of numerous example embodiments of the present disclosure will become apparent upon reading the following detailed description of the numerous example embodiments of the present disclosure, when taken in conjunction with the appended drawings, and provided claims.

### BRIEF DESCRIPTION OF THE DRAWINGS

Further aspects, features and advantages of the present disclosure will become apparent from the following detailed description when taken in conjunction with the accompanying figures showing the illustrative numerous embodiments of the present disclosure in which:

FIG. 1 is an illustration of an example endoscope having a stretchable sheath, according to first example embodiment of the disclosure.

FIG. 2 is an illustration an example endoscope having a stretchable sheath including an optional handle, according to a second example embodiment of the disclosure.

FIG. 3(A) is an end view of the distal end of the drawn multilumen sheath assembly, according to an aspect of the disclosure

FIG. 3(B) is a side cross-sectional view A-A of a drawn multilumen sheath assembly (imaging core not shown), according to an aspect of the disclosure.

FIG. 3(C) is an end view of the multilumen sheath assembly, according to an aspect of the disclosure

FIG. 3(D) is a side cross-sectional view A-A of a multilumen sheath assembly (imaging core not shown), according to an aspect of the disclosure.

FIG. 4(A) is an end view of the distal end of the drawn multilumen sheath assembly, according to an aspect of the disclosure.

FIG. 4(B) is a cross-sectional side view A-A of the distal end of the drawn multilumen sheath assembly, including the rotatable core, according to an aspect of the disclosure.

FIG. 5 is an illustration an example endoscope having a stretchable sheath including a removable, slide-able, gripper handle and modular guide, according to a third example embodiment of the disclosure.

FIG. 6 is a perspective cut-out view of a slide-able thumb lever that removably grips the endoscope, according to a fourth example embodiment of the disclosure.

### DETAILED DESCRIPTION OF THE DISCLOSURE

Hereinafter, various modes to implement the disclosure will be described in detail with numerous example embodiments and reference to the drawings/figures. Note that, dimensions, materials, shapes, and relative arrangement of components described in the example embodiments are to be appropriately modified in accordance with a configuration of an apparatus to which the disclosure is applied and various conditions, and the scope of the disclosure is not intended to be limited to the following example embodiments.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative example embodiments. It is intended that changes and modifications can be made to the described example embodiments without departing from the true scope and spirit of the subject disclosure as defined by the appended claims.

Accordingly, the foregoing merely illustrates the principles of the disclosure. Various modifications and alterations to the described exemplary embodiments will be apparent to those skilled in the art in view of the teachings herein.

### First Example Embodiment of Endoscope with Stretchable Sheath

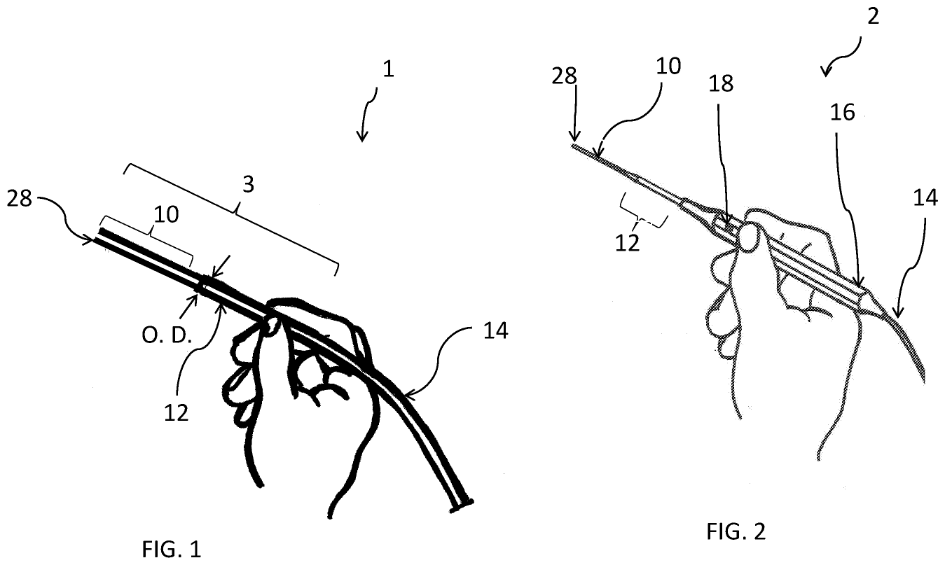

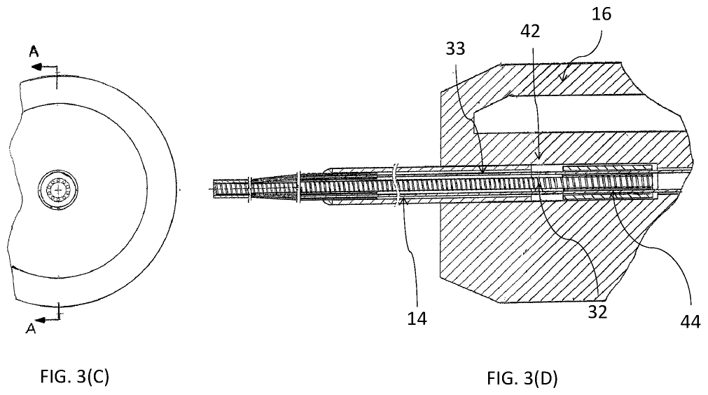

FIG. 1 is an illustration of an example medical apparatus such as an endoscope 1 having a sheath 3 that includes an elastomeric section (a stretchable sheath 14 (also referred to as “back tube”) with no handle or guide, according to a first example embodiment of the disclosure. A usable/deployable distal portion 10 of the endoscope 1 is formed and/or constructed at a distal end 28 of the example endoscope 1. The usable/deployable distal portion 10 is drawn down to a final diameter FD (see FIGS. 3(A)-3(D); further details discussed later) from an original diameter OD (25) segment 12 of a multilumen extrusion portion 22 of a drawn multilumen sheath assembly 3 of the endoscope 1. Furthermore, the elastomeric section of the sheath 14 is designed/configured to fit loosely over the proximal end of the endoscopic device 1, connecting the drawn multilumen sheath assembly 3 to the handle, while providing a small force to hold the distal optics close to the distal imaging window via its elastomeric nature. (see FIGS. 3(A)-3(D); further details discussed later).

The endoscope 1 with stretchable sheath 14 utilizes the compliant nature of certain well-known, biocompatible materials that are available in an array of durometers, with the lower durometers comprising elastic, rubbery polymers that can stretch quite a bit within their elastic limits before entering into plastic deformation which is not recoverable. This component helps keep the design simple and low cost, while providing a small force to keep the illumination optics 36 pressing against the window 24 during operation (see FIGS. 3(A)-3(D); further details discussed later).

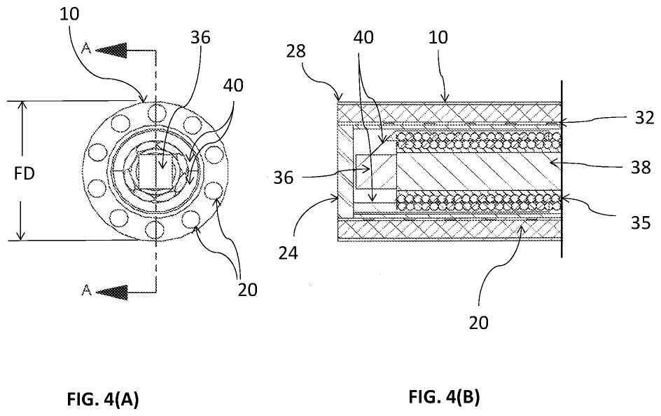

The stretchable sheath 14 may be, for example, extruded from a low durometer, stretchable material such as PEBAX 2533, a 25D durometer material on the Shore D hardness scale, or an even more conformal material such as silicone rubber to reduce the forces exerted by the imaging core on the distal window of the sheath. This material allows the stretchable sheath 14 to effectively stretch to keep the distal tip of the elongate core, which is shown in this embodiment as a rotating imaging core (see FIGS. 4(A) and 4(B) wherein the elongate core includes distal optics 36, bumper 40, drive cable 35, and illumination fiber 38, all of which integrally rotate together during operation of the endoscopic device 1; further details discussed later) in contact with the imaging window 24 (see also FIG. 4(B); further details discussed later) so that the distal optics 36 are kept at the optimal distance from the distal window 24 during normal handling and imaging. The distal optics 36 are disposed within or on the tip of the rotating imaging core 35, 36, 38 40 so that a fluoroplastic such as PTFE or FEP Teflon component or coating which remains in contact with, and rubs against the distal window 24. The distance from the distal optics 36 to the distal end of the bumper 40 (or also referred to as functionalized distal housing 40) are set accurately in manufacturing such that the bumper 40 rubs against the window 24 during operation, while the distal optics 36 are held at the optimal distance from the distal window 24.

To make the assembly process easier, the endoscope 1 sheath length is constructed into two or more segments, each with its separate function and custom construction based on its functional needs. The usable/deployable distal portion 10 (or also referred to as “working segment 10”) of the device may be 2 cm-300 cm in length. Moreover, since it is difficult to advance thin, delicate optical fibers 20 (see also FIG. 4(B); further details discussed later) through a multilumen extrusion portion 22 having a plurality of long lumens, one can minimize the length that requires this operation based on the usable length required by the application.

The elastomeric section of the sheath 14 that enables this design configuration mates with other segment(s) of the sheath that forms the usable length of the device, which is on the order of 2-18 inches (10-457 cm). For example, in the case of Ear, Nose and Throat (ENT) physicians, the usable length can be as short as several centimeters. This sheath section may be semi-rigid. For other applications or areas of interest within the body, such as URO, GI, biliary, etc., this usable length might be significantly longer, such as up to 2 or 3 meters if the scope is intended to be used for example in the GI tract through a conventional endoscope.

The elastomeric section, or stretchable back tube 14 design is optimized with an inner positioned, smooth or lubricious ID thin-walled inner tube 32 that is allowed to float and slide freely within limits inside the stretchable sheath. One preferred material that provides the smooth surface is a polyimide. This inner tube allows the elastomeric section 14 to stretch and bend, thereby conforming to minor length variations, whether they are from manufacturing tolerance stack-ups (or “axial interferences”) or from small length variations during use and flexure by the user.

It is further noted that the inner tube 32 may in fact be utilized to limit the stretchiness of any of the endoscopic devices disclosed within the subject application. In this case the functions of the inner tube 32 with at least one rectangular wire 33 coiled around the outer diameter are (but not limited thereto): (1) to provide an accurate inner diameter with lubricious surface that allows the imaging core to rotate freely, even when the endoscope 1 is bent into a tight radius during use; (2) coiled wire serves to keep the central, imaging core lumen open and round during flexure to facilitate accurate rotation and minimize Non-Uniform Rotation Distortion (NURD); this is affected by the flat metal wires 33 that are wound around the inner tube 32; (3) to provide a close-fitting lumen around the imaging core that supports NURD free imaging; and (4) to act as a length-limiting element when the elastomeric section 14 stretches.

It is also desirable to minimize any tendency for the inner tube 32 to ‘wind-up’, so a coiled flat wire 33 may also be utilized within the wall of the inner tube 32 component to resist the tendency to wind up due to torque transferred from the rotating imaging core. The proposed fabrication methods, being via multi-lumen extrusion or dip-cast, produce a thin-walled sheath component that allows the use of served or coiled wires within the wall. These metal wires may be required to help keep the inner diameter round and open during flexure, a vital requirement in an imaging system that utilizes a rotating optical element positioned within the stationary sheath component. Thus, in the inner (polyimide) tube 32, the coil serves to resist twisting from torque transferred from the rotating, imaging core.

Another aspect of the disclosure is the use of another mechanism already incorporated in the imaging core assembly which provides an additional benefit related to the stretchable elastomeric section 14 and the bumper 40 configuration. At the extreme proximal end of the device, an optical connector (not illustrated) is fixedly attached to the central, illumination optical fiber 38 that delivers light to the anatomy. When this optical connector is attached to its mating connector in the motor or patient interface unit side of the connection system, a ferrule (not illustrated) that is affixed to the illumination fiber gets compressed against a spring (not illustrated) in the optical fiber connector 38, effectively lengthening the imaging core assembly by approximately 0.50 mm-2 mm.

The above-discussed “action” is utilized to minimize strain on the elastomeric section 14 during storage and shipping to ensure that the elastic elongation of the elastomeric section 14 does not plastically deform via creep over time. This helps minimize forces between the rotating, imaging core and the distal, imaging window 24 which could be problematic for NURD-free operation by utilizing the elastic properties of the stretchable back tube, causing this up to 1 m+ long component to stretch a very small amount which may be on the order of 0.50 mm-20 mm. This interference length between the elongate core and elastomeric section 14 could be as little as 0.10 mm, and possibly as much as 3 mm, which are both well within the elastic limits of elastomeric section 14.

As can be understood from the above description, the present disclosure provides a elastomeric section 14 for endoscopes and method of manufacturing the same which (1) minimizes unwanted light transmitted from the illumination optical fiber to the detection optical fiber; (2) keeps the rotating optics (illumination) in ideal position with respect to the distal imaging window to facilitate optimal imaging, including maximized field of view; and (3) minimizes the force exerted on the imaging window during operation, to protect both the window and also the illumination optics from damage and from piercing through the imaging window of the sheath.

The proposed invention allows the stretchable sheath 14 to stretch within its elastic limits to: (1) minimize costs associated with controlling lengths of both sub-assemblies adequately; (2) keep the distal optics 36 positioned close to the imaging window 24 without crashing into the imaging window 24 with minimal forces between rotating core and imaging window 24; (3) minimize negative effects from varying distance and forces between rotating, imaging core tip (bumper 40) and imaging window 24 during catheter use and handling; and (4) minimizes the chances of the stretchable sheath 14 normalizing/annealing during shelf-life storage and shipping conditions.

### Second Example Embodiment of Endoscope with Stretchable Sheath and Optional Handle

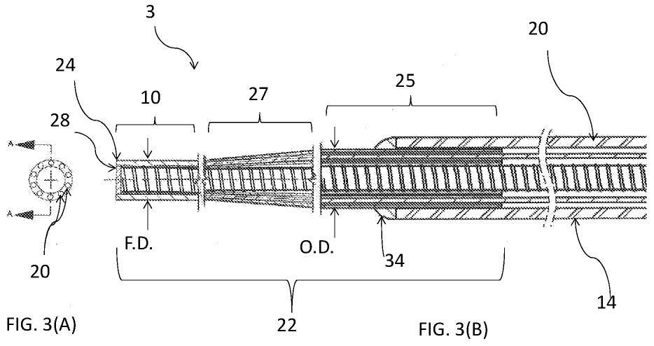

FIG. 2 is an illustration an example endoscope 2 having a stretchable sheath 14 including an optional handle 16, according to second example embodiment of the disclosure. A usable/deployable distal portion 10 of the endoscope 2 is formed and/or constructed at the distal end 28 of the example endoscope 2. In this embodiment, the stretchable sheath 14 slides over the endoscopic device 2 between the usable portion 10 and the proximal handle/connector 16. An optional vent hole 18 may also be formed in the handle 16 to prevent pressure buildup in the stretchable sheath 14.

Still referring to FIG. 2, similar to the first embodiment, the usable/deployable distal portion 10 is drawn down to a final diameter FD (see FIGS. 3(A)-3(D); further details discussed later) from an original diameter OD (un-drawn) segment 12 of a multilumen extrusion portion 22 of a drawn multilumen sheath assembly 3 (see FIGS. 3(A)-3(D); further details discussed later) of the endoscope 2. This usable, low-profile working segment 10 of the scope is attached to the stretchable sheath 14, which terminates in the handle/connector 16 at the proximal end of the stretchable sheath 14. It is further noted that the working segment 10 of the scope sheath 14 is fixedly attached to the inner tube 32 that runs the full length of the endoscopic device 2.

FIG. 3(B) is a side cross-sectional view A-A of a drawn multilumen sheath assembly 3 (elongate core not shown), according to an aspect of the disclosure. The drawn multilumen extrusion portion 22 includes the usable/deployable distal portion 10, a tapered portion 27, and an original diameter portion 25 which is not drawn down. Additionally, a plurality of optical detection fibers 20 extend along the entire length of the endoscopic device 2, but are housed within the satellite lumens of multilumen extrusion 12 for a fraction of the overall length of the device. In the example embodiment illustrated in FIGS. 3(A)-3(D), the drawn multilumen extrusion portion 22 before being drawn starts at about 4″ long having the original diameter OD, and is drawn down to the final diameter FD and approximately 7″ length. The usable/deployable distal portion 10 in the example embodiment illustrated in FIG. 3(B) is drawn down to a usable length of approximately 4″, but this disclosure is not limited to such length and instead may be a different length depending of the specific application of the endoscopic device 3. As a result of being drawn down to the final diameter FD, all clearances with respect to the optical detection fibers 20 and the usable/deployable distal portion 10 of the multilumen extrusion portion 22 are eliminated as shown in the cross-section AA of FIG. 3(A) of the usable/deployable distal portion 10.

Still referring to FIG. 3(B), positioned at the distal end 28 of the usable/deployable distal portion 10 of the endoscope 1 is the distal imaging window 24 (See also FIGS. 4(A)-4(B). The imaging window 24 may be glass or plastic and can be integrally formed or inserted, formed from, or melt-bonded into the native multilumen extrusion portion 22 material. Hence, the imaging window may be made from the native extrusion material (integral), or inserted and held in place using adhesive or melt bond. Furthermore, the integral distal imaging window 24 is used for illumination only and therefore does not cover the optical detection fibers 20.

Still referring to FIG. 3(B), the elastomeric section of the sheath 14 is attached/bonded to the handle/connector 16 via adhesive or epoxy. Similarly, the elastomeric section 14 is attached/bonded via adhesive or epoxy at an attachment point 34 to the un-drawn, original diameter portion of multilumen extrusion portion 12.

Referring to FIG. 3(C), a distal end view of sheath 3 including handle 16 is shown. FIG. 3(D) shows cross-sectional side view A-A of sheath 3. The elastomeric section 14 design uses an inner positioned, lubricious ID, thin-walled inner tube 32 (here, a polyimide tube including at least one helical wire) is allowed to float and slide freely within limits inside the handle/connector 16 and is not fixed and/or bonded to the handle 16 so it allows the elastomeric section 14 to stretch, keeping the rotating core lightly pressed against the distal window. Inner tube 32 is has an adhesive-bonded hub 44 at its proximal end sized to slide freely within handle 16 within diameter 42 to limit the amount of stretch of elastomeric sheath 14, thereby allowing sheath 3 to conform to minor length variations, whether they are from manufacturing tolerance stack-ups (or “axial interferences”) or from small length variations during use and flexure by the user. To enhance such desired effect, the elastomeric section 14 is constructed of a low-durometer, stretchable material such as PEBAX, silicone rubber or polyurethane with durometer between 40A and 40D on the Shore D hardness scale. This allows the elastomeric back tube 14 to stretch and conform to the length of the “rotating core” (discussed in further detail in FIGS. 4(A)-4(B) that is pressed lightly against the distal imaging window 24, thereby minimizing the forces and therefore friction between the two components.

FIG. 4(A) is an end view of the distal end 28 of the usable/deployable distal portion 10 of the drawn multilumen sheath assembly 3, and FIG. 4(B) is a cross-sectional side view A-A of the distal end 28 of the usable/deployable distal portion 10 of the drawn multilumen sheath assembly 3, according to an aspect of the disclosure. As discussed earlier and further illustrated in FIG. 4(B), positioned at the distal end 28 of the usable/deployable distal portion 10 of the endoscope 1 is the distal imaging window 24. Positioned internally within the usable/deployable distal portion 10 of the endoscope 1 is a bumper component 40 that secures distal optics 36 proximate the imaging window 24. Distal optics 36 are held within bumper component 40 with adhesive, and are held near the imaging window 24, and connected to distal optics 36 is an illumination fiber 38 which extends the entire length of the endoscopic device 2. As shown in FIG. 4(B), the bumper component 40 has an angle-cut distal end (or bevel edge) to allow illumination light to create a wide field of view. Moreover, the short, usable, low-profile working segment 10 of the endoscope 2 is fixedly attached to an inner polyimide tube 32 including a coiled flat metal wire 33 (see FIG. 3(B)) that runs the full length of the endoscopic device 1.

Still referring to FIGS. 4(A)-4(B), the “rotating imaging core”, which is positioned concentrically inside the inner polyimide tube 32 may be considered to include the bumper component 40, the illumination fiber 38, drive cable 35 and distal optics 36, all of which integrally rotate together during operation of the endoscopic device 2. As an example, the drive cable 35 includes at least one rotatable coiled wire circumferentially coiled around the illumination fiber such that the at least one rotatable coiled wire is fixedly secured to the rotatable illumination fiber such that the rotatable illumination fiber and the at least one rotatable coiled wire rotate as together. A preferred embodiment of the disclosure calls for the rotating imaging core 35, 36, 38 and 40 to operate while pressing against the imaging window 24 at the distal tip 28 of the endoscopic device 2, which keeps the distal optics 36 at an optimal distance from the window 24 to maximize the field of view and minimize unwanted reflections from the window 24 surfaces. This is achieved by the utilization of the bumper 40 component that is constructed of a low friction material such as Teflon, or a coating with low-friction to avoid excessive friction which could negatively affect NURD performance.

Still referring to FIGS. 4(A)-4(B, the distance from the distal optics 36 to the distal tip of the bumper 40 surface is set during manufacturing and held in place with epoxy or adhesive. It is desirable to minimize forces between the bumper 40 and window 24 to effectively minimize negative frictional effects from the same, while keeping the imaging core tip in contact with the imaging window 24 at the distal end 28 of the endoscopic device 2. A very small force is required to keep the bumper 40 pressed against the imaging window 24 during operation, and an axial interference on the order of 0.10 mm and 10 mm between the overall length of the imaging core and the sheath assembly 14 keeps the imaging core in contact with the window 24 via the elastic deformation of the elastomeric section 14 segment.

Still referring to FIG. 4(A), the end view of the distal end 28 of the usable/deployable distal portion 10 drawn down to the final diameter FD and further showing the plurality of optical detection fibers 20 which extend along the entire length of the endoscopic device 2. As has already been discussed, as a result of being drawn down to the final diameter FD, all clearances with respect to the optical detection fibers 20 and the usable/deployable distal portion 10 of the multilumen extrusion portion 22 are eliminated from the usable/deployable distal portion 10 (as shown in FIG. 4(A)). In the example embodiment shown in shown in FIG. 4(A), ten detection fibers 20 are inserted into the satellite lumens, however, it is noted that the disclosure should not be limited to ten detection fibers 20. And in other alternative embodiments, one or more satellite lumens can be utilized for flushing/washing the window 24, drainage, and/or aspiration.

As one who is experienced in the art will recognize, as an operator flexes a hollow, cylindrical member, the effective length changes as a function of bend radius. This greatly complicates the use of the above bumper 40 design, because different catheter positions and shapes can generate varying forces between the bumper 40 and window 24, which could negatively impact NURD performance. Accordingly, an aspect of the disclosure is to minimize such complications.

### Third Example Embodiment of Endoscope with Elastomeric Section of the Sheath and Optional Removable, Slide-Able Handle, Gripper Handle and Modular Guide

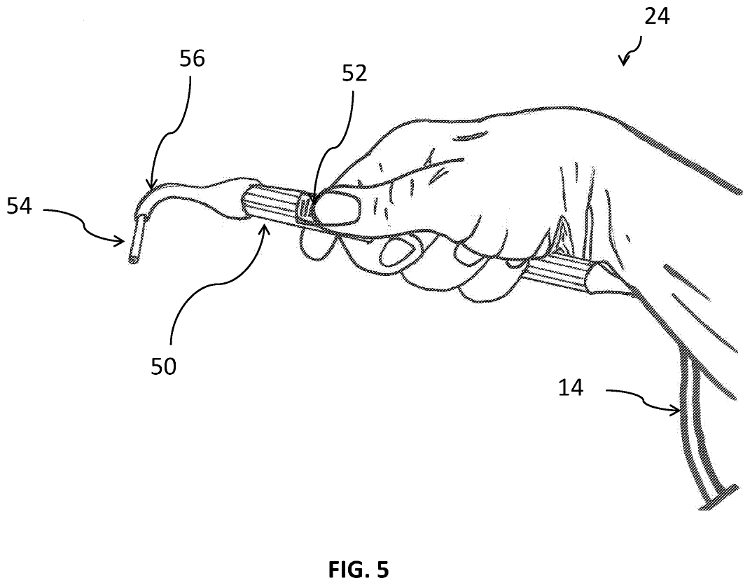

FIG. 5 is an illustration of an example endoscope 5 having an elastomeric section 14 where the endoscope includes a removable, slide-able, gripper handle 50 and modular guide 56, according to a third example embodiment of the disclosure. The sliding handle 50 may be fitted with a variety of rigid and/or semi-rigid guides 56, such as the maxillary sinus guide. Moreover, the distal drawn down working segment 54 of the endoscope 5 may also be semi-rigid. A thumb lever 52 may be included which is integrated within the modular, slide handle 50 which when pushed forward advances the endoscope 5 with respect to the guide 56. The endoscope 5 removably fits into the handle 50, which grasps the endoscope 5 with the slide-able thumb lever 52 so the drawn down working segment 54 is effectively extended through the modular guide 56, and can also be extended beyond the modular guide 56.

Still referring to FIG. 5, the slide-able handle 50 is configured to slidably grip the endoscope 5 at a junction between the distal drawn down working segment 54 and the elastomeric section of the sheath 14. This area is constructed with a thicker wall because the elastomeric section 14 overlaps the un-drawn multi-lumen extrusion. The modular, removable handle allows the endoscope 5 to be used alone or with the handle 50, which is adapted to fit existing modular guides 56 of various shapes and sizes to facilitate easy and quick access to the sinuses. Thus, the guide's 56 shape and the distal drawn down working segment 54 of the endoscope allow easy access to the sinuses.

The modular guides 56 fit onto the modular handle 50 via compliant polymer materials used in either the guide 56 or the modular handle 50, so that the guides 56 mount onto the handle 50 and are held in place with the friction of the materials, which are designed to slide, but are also somewhat tacky so the guide stays in place. In other alternate embodiments, the guides 56 are rigidly attached to the modular handle 50 via other means such as locking shapes, adhesives, etc. Moreover, the modular components are injection moldable to keep costs to the minimum.

### Fourth Example Embodiment of Endoscope with Elastomeric Section and Optional Removable, Slide-Able Thumb Lever Grips

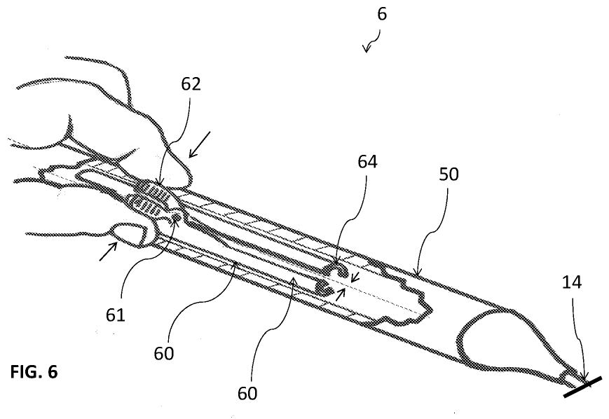

FIG. 6 is a perspective cut-out view of an endoscope 6 featuring a slide-able thumb lever 6o that removable grips, according to another aspect of the disclosure. The slide-able thumb lever 6o removably grips the scope via its split, sprung design as shown in FIG. 6. To open the grip 64, the operator squeezes the split lever 62 laterally, and the squeezing action opens the grips 64 to allow the endoscope 6 to be inserted from the proximal end. Releasing the split lever 62 closes the grip 64 onto the endoscope 6 via spring force. At that point, the user may choose to add a dedicated, existing sinus guide 56 to the distal end of the modular handle 50, as shown above, to help guide the endoscope 6 to the site of interest within the sinuses. In some preferred embodiments, the sinus guide 56 and usable portion of the endoscope 54 may be user shapeable.

### Fifth Example Providing Various Embodiment of Endoscope with Elastomeric Section Having Forward-Viewing Capabilities

A semi-rigid, low profile usable segment is drawn-down from original extrusion diameter. A distal imaging window can be bonded in place at the distal end of the usable segment or integrally formed with usable segment material. The distal imaging window is attached such that it does not allow reflected light to enter detection fibers.

Proximal to the usable segment, a larger, original multi-lumen extrusion segment is found. At the distal end, a user shapeable, modular guide tips allow easy access for the endoscope, such as access to various areas of the sinuses. These guide tips can be removably attachable to either the usable segment or a handle.

In some embodiments, the core rotates inside of a full-length inner tube with at least one wire wound around OD or within wall affixed in central lumen of multi-lumen extrusion. A stretchable outer tube is attached to original diameter multi-lumen segment to form the elastomeric section and allows the core to lightly press against imaging window. In some embodiments, the inner tube limits overall stretch of device via floating hub in handle.

In some embodiments, a dual-layer, multifilar drive cable rotates in the inner tube to provide real-time, live image. Distal optics may be held at predetermined, optimal distance from imaging window via Teflon or another low-friction coated component. The imaging core may be fitted with a standard, spring-loaded fiber optic connector to aid in connection to the motor/patient interface unit. In some embodiments, a multimode illumination fiber is disposed within the drive cable of the imaging core which is optically connected to distal optics at the distal tip of the imaging core.

Thus, the catheter as described herein has multiple advantageous properties, including, but not limited to: being forward view, having a low-profile usable segment, having a user shapeable usable length, having an easy, atraumatic access to tortuous locations (e.g., the sinuses) via shapeable, modular guide tips. It can have a semi-rigid, shapeable working length and it can be capable of operating in very tight bends on the order of 4 mm radius.

### Notable Advantageous Features and Alternative Embodiments

Low Durometer, Elastic Tube Used for Stretchable Back Tube—

A sheath segment 14 that can stretch within its elastic limits to accommodate small variations in the length of the underlying component, in this case, the imaging core sub-assembly. These length variations could be from the manufacturing tolerance, or from handling and flexure of the scope, which slightly changes the effective length of the sheath.

Low Friction Surface at Distal End of Elongate Core—

The bumper 40 design is preferably used with a low friction surface on the extreme distal tip of the imaging core that contacts the distal imaging window 24 and rubs against the inner surface of the window during rotation imaging. This controls the distance between the distal optics 36 and imaging window 24, which is critical to maintain the desired wide field of view and also minimizes unwanted reflections from the window surfaces that could degrade the quality of the image.

Floating Inner Sheath—

In some embodiments, the polyimide tube 32 is not fixedly attached at the handle 16, but is allowed to float to conform to length variations, both from manufacturing tolerances and also, and more importantly, to compensate for variations in sheath 14 length as the device is bent during use. Polyimide tube 32 is slideably attached to the handle with a slidable hub that is captured in the handle to limit the stretch of the device during handling by limiting the amount of stretch to facilitate ease of use. The hub (not shown) slides to accommodate the stretchiness of the back tube, but said stretchiness is limited by the captured hub. The elastomeric section 14 is attached to the handle 16, and is 0.50-5 mm shorter than the corresponding length of imaging core rotating within the reinforced polyimide tube 32.

Low-Friction Components or Coatings—

The utilization of a rotating core to facilitate imaging is preferably used with a low-friction inner window surface to minimize frictional resistance that could generate Non-Uniform Rotation Distortion (NURD), or, more conveniently, one or both rubbing components are equipped with a low-friction coating.

Modular Handle Components—

In some embodiments, a modular handle is provided. This provides additional capabilities and flexibility for use in multiple types of procedures. The modular handle 50 as exemplified has a slide-able grip 6o that moves the scope axially within the handle 50. The grip is spring loaded for easy, intuitive operation. The grip is split into two mating components, each with a grip segment 64 and a lever segment 62. Located between the grip and lever, the pivot 61 includes a stainless pin or wire, and a compression or torsional spring provides adequate grip force to securely hold the scope during advancement, retraction and use. Modular handles can be added upon user preference, providing additional capabilities. The handle may be fitted with vent hole to allow limited aspiration capability. The handle may be fitted with luer fitting to allow injection of rinsing solutions, drugs or other fluids for both drug or fluid delivery and for aspiration. This capability also provides in-situ window-cleaning capability via a wash fluid by utilizing one or more of the satellite lumens of the multilumen extrusion. Preferably, the modular handle quickly attaches to the device to enhance user capabilities. The modular handle component may be comprised of, for example, nylon, ABS, Delrin, PC, or any commercially available polymer that can be formulated to be semi-rigid or rigid and formed to a handle shape.

The handle may include a vent hole formed therein the handle sub-assembly. The handle may be slidably attached to the outer tube at a position proximate the proximal end of the first extrusion segment; and a slide-able thumb lever is integrated into the handle sub-assembly configured to grasp the outer tube, wherein when the thumb lever is pushed forward towards a distal end of the handle sub-assembly, the endoscope is advanced forward, and when the thumb lever is pulled backwards towards a proximal end of the handle sub-assembly, the endoscope is retracted. Further, a modular guide may be removably attached to the distal end of the handle configured to guide a semi-rigid second extrusion segment. In some embodiments, the medical device having a slide-able thumb lever comprises a pair of opposing slide-able grips interconnected by a spring loaded fulcrum.

Additionally, modular guide tips quickly attach to usable segment or to handle. These user-formable modular guide-tips for various areas within the sinuses. Axial advancement through the modular guide tips can be indicated.

Drive Cable—

In some embodiments, the drive cable is a multifilar, multilayer stainless steel wires arranged to efficiently transmit angular position from proximal to distal end.

Connections—

Some embodiments include spring-loaded fiber optic connector that compresses slightly when attached to mating connector. These connectors can effectively lengthen the imaging core by approx. 0.50 mm-1.00 mm.

### Other Aspects, Features, Fabrication, and Example Embodiments

According to an aspect of the embodiments of the disclosure, assembly considerations are taken into account because the device requires several thin, delicate optical fibers to be inserted into the satellite lumens of the distal, usable length of the segment that forms the working end of the sheath. These detection fibers collect light reflected and/or emitted from the anatomy and transmit this low intensity light efficiently to the spectrometer or detector that is housed in the system box or cart. Since these delicate optical fibers cannot easily be inserted far into their lumens within the multilumen extrusion, the drawn-down, usable length is minimized, and the remainder of the device outer sheath length is formed of an elastic material such as silicone rubber, low-durometer PEBAX copolymer, urethane, etc., with detection fibers inside of the elastomeric tube. This remaining length of the device need not be ultra-low profile because it does not need to be inserted into the body or modular guide.

According to yet another aspect of the embodiments of the disclosure, the usable portion referenced above is the only segment of the device whose diameter must be minimized, to both allow the device to enter smaller lumens, spaces or gaps within the anatomy, and also to minimize any trauma associated with its use based on its size/diameter. Additionally, the user may desire that the scope is formable, an attribute that is better achieved through the elimination of clearances required for assembly. For example, the optical fibers require clearance for insertion into their satellite lumens. And, the draw down process eliminates those clearances required for assembly, and also creates a formable tip because the optical fibers within the satellite lumens are allowed to slide with respect to the sheath due to their smooth surface.

According to other aspects of the embodiments of the disclosure, one or more of the proposed fabrication methods allows the design of a catheter sheath with very thin walls and zero clearance around inserted items. This is an important factor in the design to keep the outer profile within the desired limits.

Additionally, according to yet other aspects of the embodiments of the disclosure, thin-walled tubes generally deform from original round shape to oval when bent, and if the radius of curvature is small enough the tube will kink. Whether ovalized or kinked, the inner diameter no longer allows the imaging core to rotate freely, and image distortion called Non-Uniform Rotation Distortion (NURD) will likely occur. To combat this tendency, a reinforced tube has been included at the inner diameter in some embodiments. This PTFE lined polyimide tube is fitted with one or more metallic coils that serve to keep the inner diameter open and round through tight bends, a critical feature for devices that need to operate around and through tight bends with radii on the order of 10 mm or less.

Thus, in some embodiments, the multilumen segment is comprised of PEBAX, PVC, polyurethane, or similar thermoplastic material. The inner tube is comprised of polyimide, PEEK, nylon, urethane, PVC, PC or similar material with lubricious, low friction properties on the inner diameter. The stretchable segment may be comprised of, for example, PEBAX, PVC, polyurethane, silicone rubber or similar conformal material.

And, according to another aspect of the numerous embodiments of the disclosure, a preferred embodiment is provided which utilizes said multi-lumen extrusion is extruded with larger lumens and outer diameter and then drawn down to final outer diameter using a heated drawing die, or shrunk using heat shrink tube to its final preferred outer diameter using heat and tension, to size the outer diameter, again with heat. This allows the extruder to manufacture the original, larger diameter tubing with thicker wall thicknesses and more generous tolerances, which makes the extrusion easier to fabricate and keeps costs down.

Additionally, according to another aspect of the embodiments of the disclosure, the drawing-down process utilizes an appropriately sized mandrel to support the inner lumen diameter. A PTFE coated steel mandrel which is then removed after drawing down serves to keep the inner diameter open and round during draw-down to maintain the desired level of performance with respect to imaging requirements and particularly NURD. In this case, the polyimide tube is disposed between the multilumen extrusion and the central mandrel, but the mandrel functions the same way as listed above.

Moreover, according to another aspect of the embodiments of the disclosure, the distal, usable portion of the device is drawn down or shrunk using heat shrink tubing to minimize or in some cases eliminate clearances between the inserted components and the native material of the multilumen extrusion, thereby minimizing the resulting outer diameter. In a preferred embodiment, the imaging core, and a PTFE-lined inner polyimide tube with metallic coil and several detection fibers are needed at a minimum to get the performance required by the application, which is generally related to medical imaging catheters and endoscopes. The rotating, imaging core is disposed within the central lumen inside the PTFE-lined polyimide tube, while sharing the same centerline as the multilumen extrusion.

And, according to another aspect of the embodiments of the disclosure, a preferred embodiment of this disclosure calls for the imaging core to operate while pressing against the imaging window at the distal tip of the device, which keeps the distal optics at the optimal distance from the window to maximize the field of view and minimize unwanted reflections from both window surfaces. But, as one who is experienced in the art will recognize, as one flexes a hollow, cylindrical member the effective length changes slightly as a function of bend radius. This greatly complicates the use of the aforementioned design, because different catheter positions and shapes can generate varying forces between the imaging core and distal window as the effective length of the sheath changes due to bending, which could negatively impact NURD performance if the sheath were unable to conform to the minor variations in length due to bending.

It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

The subject disclosure relates to U.S. Patent Appl. Pub. 2017/0290492, entitled “ENDOSCOPE SHEATH WITH INTEGRAL IMAGING WINDOW” the content of which each application is expressly incorporated by reference herein in its entirety.

While the disclosure has been described with reference to example embodiments, it is to be understood that the present disclosure is not limited to the disclosed example embodiments. The scope of the following claims is to be accorded the broadest interpretation so as to encompass all such modifications and equivalent structures and functions.

What is claimed:

1. A medical apparatus comprising:
an elongate core having a distal and proximal end;
a sheath substantially surrounding the elongate core, the sheath comprises an elastomeric section which is concentrically disposed and is configured to allow the length of the sheath to conform to the length of the elongate core; and
a window attached to the distal end of the sheath, wherein the distal end of the elongate core is near the window.

2. The medical apparatus of claim 1, wherein the elastomeric section of the sheath comprises a stretchable thermoplastic elastomer.

3. The medical apparatus of claim 1, wherein the elastomeric section is extruded from one of PEBAX, silicon rubber or polyurethane.

4. The medical apparatus of claim 1, wherein the sheath further comprises a semi-rigid section.

5. The medical apparatus of claim 1, wherein the window is embedded in the sheath.

6. The medical apparatus of claim 1, wherein the elongate core comprises a rotatable imaging core.

7. The medical apparatus of claim 1, wherein the distal end of the elongate core is maintained within 0.1 mm of the window.

8. The medical apparatus of claim 1, wherein the distal end of the elongate core is touching the window.

9. The medical apparatus of claim 1, wherein the contact force between the window and the elongate core is low.

10. The medical apparatus of claim 1, wherein the elastomeric section of the sheath is near the distal end of the sheath.

11. The medical apparatus according to claim 10, wherein the handle sub-assembly includes a vent hole.

12. The medical apparatus of claim 1, wherein the sheath further comprises an inner positioned tube having a smooth, lubricious inner surface.

13. The medical apparatus of claim 12, wherein the inner positioned tube has at least one coiled wire, the elongate core configured to be freely rotatable within the inner positioned tube.

14. The medical apparatus according to claim 1, further comprising: a handle sub-assembly detachably attached to the elongate sheath.

15. The medical apparatus according to claim 1, further comprising a spring loaded connector, the connector configured to stretch the sheath when connected.

16. An endoscope comprising:
an elongate core having a distal and proximal end, wherein the elongate core comprises an illumination fiber and a distal optics at the proximal end of the elongate core;
a sheath substantially surrounding the elongate core, the sheath comprises an elastomeric section which is concentrically disposed around at least a proximal portion of the elongate core and an inner positioned tube which is concentrically disposed around the elongate core; and
a window attached to the distal end of the sheath, wherein the distal optics near the window.

17. The endoscope of claim 16, the elongate core further comprising a drive cable, and wherein the elongate core is rotatable.

18. The endoscope of claim 16, wherein the inner positioned tube has at least one coiled wire, the elongate core configured to be freely rotatable within the inner positioned tube.

19. The endoscope of claim 16, wherein the elastomeric section of the sheath comprises a stretchable thermoplastic elastomer.

20. The endoscope of claim 16, wherein the distal end of the elongate core is maintained within 0.1 mm of the window or is in contact with the window.
